(19) United States
(12) Patent Application Publication
(10) Pub. No.: US 2006/0247585 A1
(43) Pub. Date: Nov. 2, 2006

(54) Disposable pads for applying and distributing substances to target surfaces

(76) Inventor: Albert R. Kelly, Douglaston, NY (US)

Correspondence Address: Evelyn M. Sommer, Suite 825, 250 Park Avenue, New York, NY, 10022, US

(21) Appl. No.: 11/274,526

(22) Filed: Nov. 15, 2005

Related U.S. Application Data
(60) Provisional application No. 60/628,171, filed on Nov. 17, 2004.

(51) Int. Cl. A61M 35/00

(52) U.S. Cl. 604/290; 604/292; 604/293

(57) ABSTRACT

The applicators, and methods for making them, their use in manually applying controllable amounts of moist, viscous lotions, creams, gels or dry powders onto target skin and hard surfaces. The applicators comprise an impervious backing sheet, a three-dimensional core layer with reservoirs in the shapes of cones or honeycombs wherein active compositions in the form of viscous lotions, gels, creams or dry powders are located, a microporous flow-directing cover sheet and an optional impervious overwrap. The applicator is capable of releasing the active composition in a controlled manner and protecting the user's hands from contacting the viscous material, to prevent potential soiling, cross-contamination and/or skin irritation. The applicators can be made to form hand or finger mitts, gloves, flat or tubular pads, wraps, facemasks, booties, and more.

This application claims priority of Provisional Application No. 60/628,171 filed Nov. 17, 2004.

### FIELD OF THE INVENTION

The present invention generally relates to the applicators, and methods for making them, their use in manually applying controllable amounts of moist, viscous lotions, creams, or gels onto target surfaces. More specifically, the present invention relates to such applicators which provide both a metering device for controlled release and a means for protecting the user's hands from contacting the viscous material, to prevent potential soiling, cross-contamination and/or skin irritation. The applicators can be made to form hand or finger mitts, gloves, flat or tubular pads, wraps, facemasks, booties, and more.

### BACKGROUND OF THE INVENTION

Generally, viscous lotions, creams, and gels intended for application to skin and hard surfaces are packaged in bottles or tubes. Such substances are dispensed either directly onto the targeted surface and spread over the desired area by hand, or first onto a pad or like applicator, which is then wiped onto the surface by hand. In such cases, the substance must then be washed or wiped from the user's hand. If the user is away from home, the user must carry the entire bottle or tube containing the substance, which can be cumbersome.

If the viscous substance is for skin care use, as a cosmetic, preventative, or medicament, and is applied by hand, particularly by a caregiver or other person's hand, there is the real potential for cross-contamination. If the viscous substance is for surface care and contains skin sensitizing agents, such as antimicrobials, allergens, solvents, or acidic or alkaline ingredients, its application by hand can cause skin irritation, especially if hands are not washed after use.

Whether the viscous substance is used for skin or hard surface care, it is important that it be applied evenly in effective amounts for each product, and that it not be under-applied to be ineffective, or over-applied, leaving a residue. Application by hand can cause either eventually.

Many viscous substances for skin care are massaged into the skin, and for surface care are used to aggressively clean or polish the targeted surfaces. Applications of these types normally require companion use of wipes, towels, or rags, which subsequently have to be cleaned or discarded after use.

The key object of this invention is to provide cost effective applicators for these high viscosity substances, in the form of a single product that largely eliminate the negatives normally associated with the use of such products as currently supplied, and help make their use easier and more effective.

For example, one object is to provide a pre-loaded applicator engineered to deliver a single dosage of a personal care substance in a format that is conveniently portable, and that can be carried safely in one's pocket or purse, especially if indicated for use away from home.

Another object is to provide a pre-loaded applicator that can be used without having to actually touch the viscous substance to be applied to the skin or hard surfaces, and that can be discarded conveniently after use without leaving a residue on any surface.

Still another object is to provide a pre-loaded applicator that eliminates or minimizes the potential for cross contamination, particularly where the viscous skin care substance is to be applied by another party, such as a caregiver, a mother, a spouse or any other person who may be a carrier or recipient of a hand-borne organism that could potentially be infectious. A related object for viscous substances used for hard surface care containing ingredients such as antimicrobials, pesticides, acids, alkali's or solvents is to eliminate or minimize the potential for skin irritation or allergic responses, that could result from extended contact with such substances, on hands and skin in general.

Yet another object is to provide a pre-loaded applicator that will help meter and control delivery of the viscous skin and surface care substances to insure an even and smooth application of an effective and safe amount of the specific product involved. The latter is a feature, which typically cannot be controlled effectively by hand.

Still another object is to provide a pre-loaded applicator that will enhance the effectiveness of skin or surface care product. For example, the contacting surface of the applicator for skin care products could be engineered to aid cleaning, exfoliating and massaging benefits. For surface care applications, the outer surface of the applicator could be engineered to aid the cleaning and polishing of those target surfaces.

A final object is to provide an applicator structure that can be configured in a variety of user-friendly formats, including wipes, pads, mitts, gloves, muffs, face masks, wraps and more.

U.S. Pat. No. 2,790,982 issued to Schneider teaches a single use applicator package comprising an impervious sheet folded onto itself to receive user's fingers, a pervious sheet and a capsule containing the active wherein the capsule is positioned between the impervious sheet and the pervious sheet.

U.S. Pat. No. 3,053,385 issued to Spees teaches a disposable applicator comprising a body of absorbent material within which a container is present holding fluid substance and the fluid can be released upon the activation of a rupturing device.

U.S. Pat. No. 3,369,267 issued to Friedland et al teaches an applicator comprising a flat foldable pad saturated with material to be applied in a wiping action, two flexible impervious sheets enclosing the pad, wherein one of the sheets possesses a detachable strip which can be removed to expose the pad. The applicator can be grasped by holding onto the impervious sheets of a folded applicator.

U.S. Pat. No. 3,608,708 issued to Storandt teaches an applicator mitt having a normally open chamber for receiving the user's hand, a treated pad, constructed of moisture absorptive material, attached to the exterior portion of said hand chamber, and protective covers jointed to said chamber.

U.S. Pat. No. 4,643,725 issued to Schlesser et al teaches a combination package and applicator comprising a substance impregnated fibrous absorbent carrier pad attached to an impermeable backing sheet, and grasping means is provided by folding the backing sheet, or attaching a handle to the backing sheet or constructing the backing sheet as a glove-like member.

U.S. Pat. No. 4,701,168 issued to Gammons teaches a disposable applicator comprising a support sheet, an absorbent pad impregnated with fluid actives, and a removable cover sheet wherein the support sheet and cover sheet encloses the absorbent pad and the removal of the cover sheet exposes the pad for release of the liquid active. Grasping means are provided by the incorporation of upright handles on the support sheet and the handles can be squeezed for purposes of both handling the applicator and for flexing the applicator so as to push the exposed absorbent pad towards the target surface.

U.S. Pat. No. 6,257,785 issued to Otten et al. teaches an applicator in the form of a glove or mitt, comprising a sheet wherein reservoirs are present for holding substances to be delivered upon compression of the sheet onto the target surface. Optionally, an additional layer of porous material can be present to provide additional protection of the substance prior to activation and may also provide additional distributive benefit to more evenly disperse the substance onto the target surface.

U.S. Pat. No. 6,315,482 issued to Girardot et al. teaches an applicator for applying and distributing a substance onto a target surface, comprising a substantially planar sheet of compressible, conformable material having discrete reservoirs extending into the interior of the sheet, said reservoirs containing actives. During use, compression of the sheet of material via an externally-applied force normal to the surface applies and distributes the active onto the target surface.

### SUMMARY OF THE INVENTION

The present invention describes a pre-loaded applicator, preferably in the form of hand or finger mitts, gloves, pads, tubular muffs, and wraps for applying and distributing a high viscosity contained therein onto a target surface.

The applicator comprises a impervious backing sheet consisting preferably of a non-woven and impervious film laminate, a three dimensional core layer incorporating multiple reservoirs for holding substances in the form of viscous lotions, creams, paste, gels, or dry powders or the like, a microporous flow-directing cover sheet and, optionally an impervious overwrap to enclose the article for protection. One surface of the core layer contains multiple reservoirs, the openings of which can be circular, square, or diamond-shaped and the reservoirs are conical or flat-bottomed with the cone points or flat bottoms in either case directed inwardly. The reservoirs hold the actives in flowable forms such as lotions, creams, or gels, which are applied by the user. Alternatively, the reservoirs hold the dry powders that are activated by water forming foaming cleanser, a moisturizing lotion or a skin or surface treatment. The impervious backing sheet, consisting of a non-woven material and impervious film laminate, is attached to the core layer with the exposed surface of the impervious film laminate material of the backing sheet in contact with the bottom surface of the core layer. The impervious backing sheet is provided to retain and protect the active during storage and use. A flow-directing cover sheet is sealed over the surface of the fully loaded, coated core layer. The cover sheet possesses porous or microporous openings whereby the flow of the active is directed outwardly and reentry of the active back into the article is prevented, to inhibit any cross-contamination in-use, among other benefits. The applicator optionally can have a removable fourth layer or overwrap placed on top of the meshed cover sheet, which is impervious to protect and enclose the exposed viscous substance. The preferred form of the article is a pad that can be used, flat, or formed into a hand or finger mitt, a muff, a wrap, or other suitable forms such as gloves.

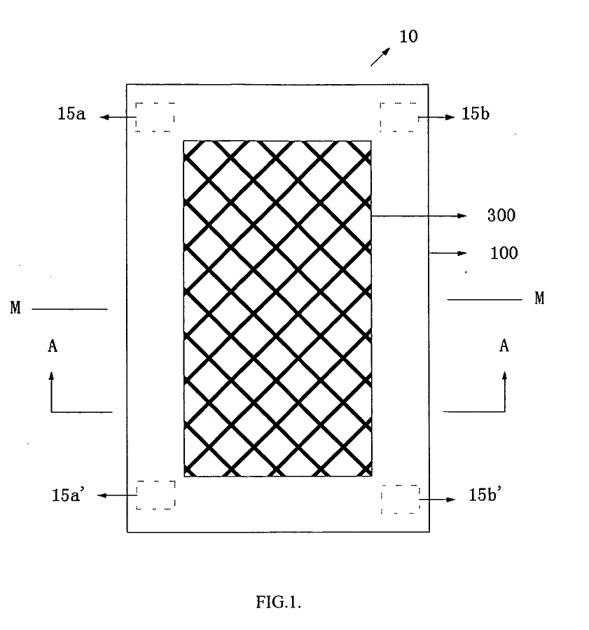

FIG. 1 is a plan view of an applicator in accordance with the present invention, in the form of a finger mitt before use.

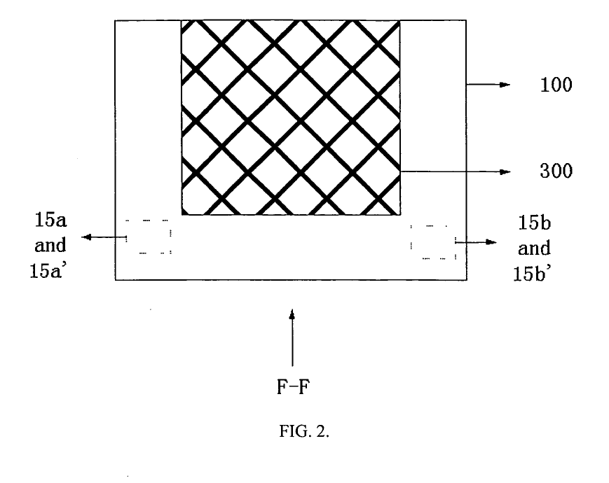

FIG. 2 is a plan view of the applicator of the invention and ready for use as a finger mitt.

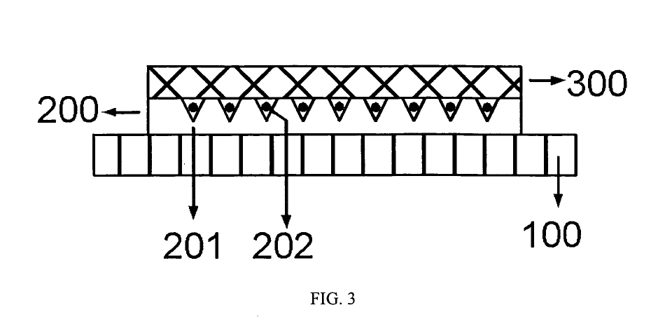

FIG. 3 is a cross sectional view taken along line A-A in FIG. 1 in a first preferred embodiment, wherein the reservoirs are in the shape of cones.

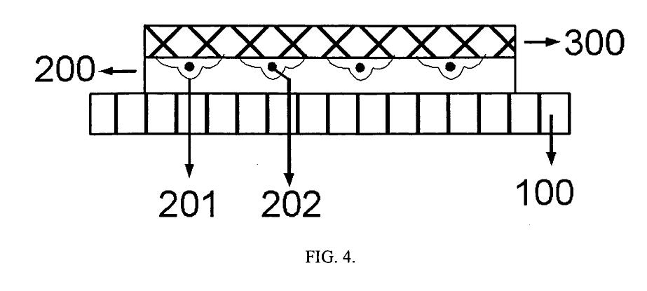

FIG. 4 is a cross sectional view taken along line A-A in FIG. 1 in a second preferred embodiment, wherein the reservoirs are in the shape of honeycombs.

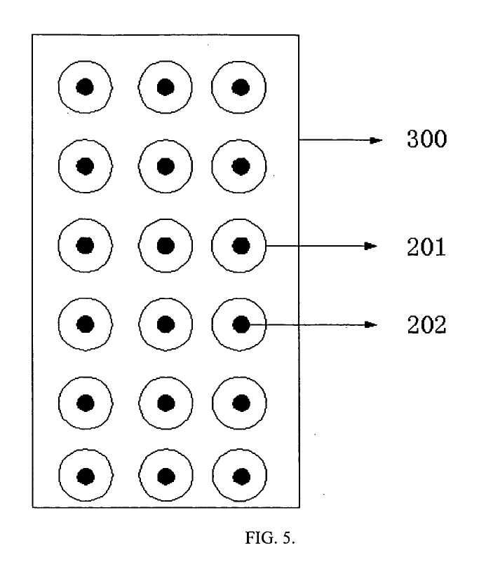

FIG. 5 is a top view of the core layer taken from the side of the reservoir openings in the first preferred embodiment, wherein the reservoirs are in the shape of cones.

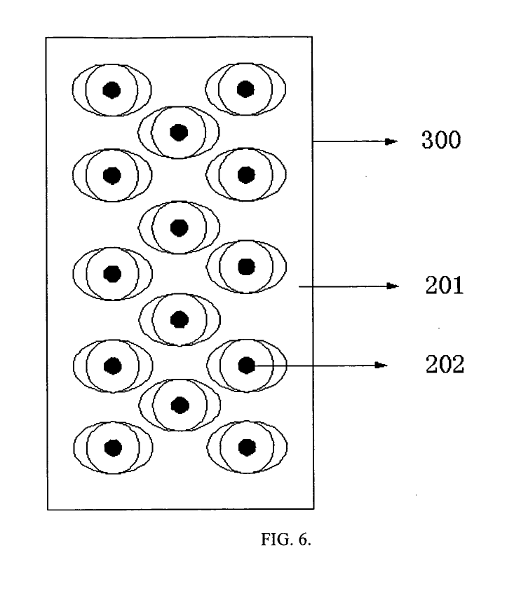

FIG. 6 is a top view of the core layer taken from the side of the reservoir openings in the second preferred embodiment, wherein the reservoirs are in the shape of honeycombs.

### DETAILED DESCRIPTION OF THE INVENTION

As used herein, the term “pad” is used to describe articles of three or more plies including a three dimensional core element loaded with a viscous substance that is dispensed through a porous cover sheet in amounts controlled by both the size and concentration of openings within a given area, and the pressure applied in use. These “pads” are intended to be disposable, and are not intended to be reused.

As used herein, the term “mitt” refers to a pad-like article, with a flexible plastic strap, under which one finger, a number of fingers, or the entire hands is inserted to hold and guide the pad over the targeted surfaces.

As used herein, the term “muff” refers to an extended rectangular pad-like article that can be formed reversibly into a tubular structure, fitting fingers or hands with the treated surfaces facing out, so that the substance can be applied to a targeted surface.

A preferred embodiment of the applicator of the present invention in the form of a mitt is the mitt shown in FIGS. 1-6 before use.

FIG. 1. is a plan view of the mitt 10 of the present invention in its packaged state before use. At each of the four corners on the side of the mitt opposite to the side of the active (the under side of the mitt shown in FIG. 1), there is a fastening means 15 (15a, 15a′, 15b and 15b′) for holding the two folds of the mitt together during use (15a with 15a′ and 15b with 15b′, FIG. 2). The holding means 15 can be any of conventional designs, such as a double-sided tape and a Velcro tacky surface. Alternatively, other fastening means can be used, such as bands. During use, one or more fingers can be inserted into the folded mitt shown by the arrow F-F in FIG. 2.

The mitt comprises at least three layers of material, as illustrated in FIGS. 3 and 4 which are cross-sectional views of FIG. 1 along the line A-A, the impervious backing sheet 100, the core layer 200 containing reservoirs 201 and the active 202 inside the reservoirs, and microporous cover layer 300. The backing sheet 100 can be of the same size compared to the core layer 200 and cover layer 300. More preferably, the backing sheet 100 is of larger size compared to the core layer 200 and cover layer 300, so that the two liquid impermeable backing sheets on the two folds of a mitt can fully enclose the core layer and the cover layer to prevent leakage of active prior to use if the mitt is stored in a folded manner with the two halves of the cover layer 300 dissected by the line M-M (FIG. 1) facing each other. The core layer 200 possesses at least one reservoir 201, preferably in the shape of cones (FIGS. 3 and 5) or honeycomb reservoirs (FIGS. 4 and 6) with the reservoirs open at the surface of the core layer, which is in contact with the microporous cover layer 300. Active 202 in lotion, cream, gel or powder form is contained in the reservoirs 201 during storage. A porous cover layer 300 is applied to the side of the core layer opposite to the backing sheet, and in contact with the reservoir openings.

The core layer is preferably a film from any synthetic or natural fibers that are conformable and flexible under the pressure to be applied under typical use. The film for the core layer is preferably vacuum formable to produce the cone or flat bottom shaped reservoirs. It is also necessary for the film to be chemically stable and to provide an effective moisture and vapor barrier to prevent evaporation or breakdown of the substances contained therein. Useful resins include, but are not limited to, polyethylene, polypropylene, PET, PVC, PVDC, latex structures, nylon, etc. Polyolefins are generally preferred due to their lowered cost and ease of forming.

The impervious backing material can be of any synthetic or natural material, such as those aforementioned, that does not allow the active composition to permeate under typical conditions encountered in storage and use of applicator. The backing material can be in the form of a film, a nonwoven material, or a combination thereof.

The microporous cover material may be of any material sufficiently porous as to not block or significantly impair the ability of the active composition to be dispensed from the core layer onto the target surface, against which the microporous cover material would be placed in use. Microporous cover material should preferably incorporate the same fibrous material used to form the three-dimensional cores, to insure that they can be bonded together by heat or ultrasonics, for optimal process ability efficiencies. Any of the aforementioned materials for core layer may be utilized. The micropores in the cover layer direct the flow of active composition in an outward manner and re-entry of the active composition back into the article's core layer is effectively prevented. The inclusion of such a microporous material provides additional protection for the substance prior to activation of the core layer and can provide significant distributive benefits to more evenly disperse the active composition onto the target surface particularly when translational motion of the application is also employed. The microporous material may have an outer surface that also provides additional benefits such as exfoliation, mild abrasivity, and lathering or foam generation, where indicated. The microporous material may also be coated with another substance for distribution, which may desirably interact with, augment, or otherwise enhance the effectiveness of the active composition in the three-dimensional core layer.

The active composition to be delivered using this disposable applicator can include any flowable substance which is substantially non-flowing prior to delivery to a target surface. The active composition can have a viscosity of at least 2,000 CPS and no more than 80,000 CPS, preferably between 20,000 and 60,000 CPS, which can be flowable under pressure, at typical body temperatures or activation, upon the combination thereof. The active composition is intended to begin to be released from the reservoirs when exposed to contact with target surfaces and when the applicators are subjected to pressure, elevated temperatures, or the combination thereof.

Alternatively, the active composition to be delivered by this disposable applicator can include powders. The active composition is intended to begin to be released from the reservoirs when exposed to contact with target surfaces, when the applicators are subjected to pressure, friction, or the combination thereof, or when wetted and exposed to contact with target surfaces.

The preferred active compositions for this disposable applicator include, but are not limited to, skin care and moisturizing lotions, sunscreens, antiperspirants, topical medicaments, cosmetics, lubricants, cleansers, baby powders, hard surface cleansers, polishers, protectants, and the like.

The treated composite structure for any of these devices can be manufactured according to the following process. The backing material and the core material are laminated together, preferably by heat, as a roll good. That roll is then unwound with the tops of the cones facing upward. The cones are filled with active ingredients including viscous lotions or creams by injection, with any excess to be doctoring off. The coated structure including the coated core material is then overlaid by the microporous cover stock and sealed peripherally onto the coated portion of composite structure. The fully treated and sealed composite is then die cut to the desired size and shape. The die-cut composite is then partially sealed and packaged within the removable over-wrap.

In the event that the article is to be formed into a hand or finger mitt, the user then removes the disposable overwrap. The user can then insert his/her fingers into the pocket (F-F direction in FIG. 2). When the user touches the target surface with the microporous cover layer and applies pressure against the target surface, the three-dimensional structure of the core layer is compressed toward a two-dimensional sheet and the volume of the reservoirs are subsequently reduced. As a result, the active is forced to exit the reservoirs, through the micropores on the cover layer and finally onto the target surface, without soiling the user's fingers.

By applying different pressure onto the article, the reservoirs are expressed to a different extent, and different amounts of active composition can be delivered at different rates. This controlled release/metering effect is further enhanced by the function of the microporous cover sheet, which directs the flow in an outward manner only and helps distribute the active composition more uniformly. When the intended use is completed and the active composition in the mitt is exhausted, the mitt can be discarded.

A preferred embodiment of forming a mitt comprises forming an enclosure which can best be described by reference the enclosure for a slice of cheese wherein the lateral edges of the enclosure are releaseably sealed and there is an overwrap which extends downwardly over the enclosure to the extent of less than 50% of the top surface and which when lifted upwardly forms a holding portion extending upwardly from the mitt formed by the enclosure.

### Examples

1. Sunscreen Pads

Sunscreens are normally comprised of relatively viscous creams or lotions that are squeezed from tubes or bottles into the user's hands, or onto the targeted skin area, and then spread by hand over the area to be protected. As such, it can become a messy and imprecise process, frequently requiring two hands (one to hold the container and the other to spread the lotion or cream as evenly as possible), and finally a towel or tissue to dry the hands.

The targeted load for the Sunscreen Pad is up to 16 grams. The pad dimension of 3.75″×4.5″ will yield a core surface of approximately 16 square inches for 16 grams of sunscreen—or effectively, 1 gram per square inch.

The molded honeycombed reservoirs of the core materials supplied by Tredegar Film Products, with the code STR 7166/x32500, were engineered to those specifications, using a 2.5 mil polyethylene film plus a nominal 0.75 mil barrier onto the male side of the molded formation of the reservoirs, and the bottoms. The molded core reservoirs were then filled with the sunscreen lotion and the excess doctored off. A 60 Hex apertured microporous polyethylene film, also supplied by Tredegar Film Products, coded STR 7165/x32502, designed to direct the flow of the sunscreen lotion in an outward direction and to prevent re-entry of the expressed lotion back into the core structure, is overlaid on top of the filled core material. The filled core material and the microporous cover stock were then sealed peripherally and die-cut to form the applicator pad measuring 3.75″×4.5″. This pad was then sealed within a barrier overwrap package, which was prepared for use by removing a portion of the overwrap to expose the lotionized surface. The portion of the overwrap which was not removed, forms a band for the user's hand to hold the pad, before and during use, without ever having to touch the lotionized surface.

In field tests, a single Sunscreen Pad of this type, proved to be a more convenient and efficient means for evenly applying an effective amount of sunscreen lotion to the entire body.

2. Skin Protectant Pads

Skin protectant lotions, creams, and ointments deliver various active ingredients, including dimethicon, zinc oxide, petrolatum (Vaseline), among others. Their use is prevalent in long-term healthcare environments where incontinence among bedridden patients can produce serious problems with skin breakdown conditions, including dicubetous ulcers, if such threatened skin is left unprotected.

In such situations, skin protectant lotions, creams, and ointments are normally applied by hand, by third party caregivers or attendants, where there is the real potential for cross-contamination to both parties.

The targeted load for the Skin Protectant Pads is up to 12 grams, but preferably between 6 and 9 grams/pad. The pad dimension of 3.25″×4.5″ yields a core surface of approximately 16 square inches for such loads, or effectively, from 0.4, 0.6, or 0.8 grams per square inch.

The molded honeycombed reservoirs of the core materials supplied by Tredegar Film Products, with the code STR7166/x32500, were engineered to accommodate the specification for a 12 gram load, using a 2.5 mil polyethylene film plus a nominal 0.75 mil barrier onto the male side of the molded formation of the reservoirs, including the bottoms. The molded core reservoirs were then filled with petrolatum and the excess doctored off.

A 60 Hex apertured microporous polyethylene film, also supplied by Tredegar Film Products, coded STR 7165/x32502, designed to direct the flow of the petrolatum in an outward direction and to prevent re-entry of the expressed gel back into the core structure, is overlaid on top of the filled core material. The filled core material and the microporous cover stock were then sealed peripherally and die-cut to form the applicator pad measuring 3.25″×4.5″. This pad was then sealed within a barrier overwrap package, which was prepared for use by removing a portion of the overwrap to expose the lotionized surface. The portion of the overwrap, which was not removed, forms a band for the user's hand to hold the pad, before and during use, without ever having to touch the lotionized surface.

In field tests, skin protectant pads of this type proved to be a more convenient and efficient means for evenly applying an effective amount of petrolatum to the threatened skin of bedridden patients.

3. Skin Moisturizing Pads

Skin Moisturizing Pads are normally comprised of relatively viscous creams or lotions that are squeezed from tube or bottles into the user's hands, or onto the targeted skin area, and then spread by hand over the area to be moisturized. As such, it can become a messy process, frequently requiring two hands (one to hold the container and the other to spread the lotion or cream as evenly as possible), and finally a towel or tissue to dry off the residue.

The targeted load for the Skin Moisturizing Pads is between 3 and 12 grams/pad. The pad dimension of 3.25″×4.5″ will yield a core surface of approximately 16 square inches for such loads or effectively, from 0.2, 0.4, 0.6 or 0.8 grams per square inch.

The molded honeycombed reservoirs of the core materials supplied by Tredegar Film Products were engineered to the specification for a 6 gram load using a 2.5 mil polyethylene film plus a nominal 0.75 mil barrier onto the male side of the molded formation of the reservoirs, including the bottoms. The molded core reservoirs are then filled with the moisturizing lotion and the excess doctored off. A 60 Hex apertured microporous polyethylene film, also supplied by Tredegar Film Products, coded STR 7165/x32502 designed to direct the flow of the lotion in an outward direction and to prevent re-entry of the expressed lotion back into the core structure, is overlaid on top of the filled core material. The filled core material and the microporous cover stock were then sealed peripherally and die-cut to form the applicator pad measuring 3.25″×4.5″. This pad was then sealed within a barrier overwrap package, which was prepared for use by removing a portion of the overwrap to expose the lotionized surface. The portion of the overwrap, which was not removed, forms a band for the user's hand to hold the pad, before and during use, without ever having to touch the lotionized surface.

In field tests, these moisturizing pads provided a more convenient and efficient means for evenly applying an effective amount moisturizing lotion to the targeted skin areas prone to dryness.

4. Antiperspirant Pads

Antiperspirants conventionally comprise waxes or thick gels that are dispensed from small bottles, called “barrels” or tubes onto the targeted skin area (underarm, etc.), to be protected. These waxes, in particular contain thickening agents which can diminish efficacy and irritate skin. The most effective and best-tolerated product form is believed to be an oil-base anhydrous cream or a lotion in the form of an oil-in-water emulsion. Pads, which are the subject of this invention, avoid the disadvantages associated with the known delivery system and are ideally suited for delivering a product in an oil-in-water emulsion format that can be used conveniently without the need to carry a significant dispenser.

The targeted load for the antiperspirant cream is between 2 and 6 grams/pad. The pad dimension of 2.75″×3.5″ which yield a core surface of approximately 10 square inches for producing loads or form 0.3 to 0.6 grams per square inch.

The molded cone-shaped reservoirs of the core materials supplied by Tredegar Film Products, under the code identification STR 5539/x31617 and were engineered to the foregoing specifications, using 1.0 mil polyethylene film plus a 0.5 mil barrier film on the bottom side of the molded cone formation. The molded cone reservoirs are then filled with the antiperspirant cream and the excess doctored off. A 40 Hex apertured microporous polyethylene film, also supported by Tredegar Film Products, code STR 7164/x3251 which has been designed to direct the flow of the cream in an outward direction and to prevent re-entry of the expressed lotion back into the core structure, is overlaid on top of the filled core material.

The filled core material and the microporous cover stock were then sealed peripherally and die-cut to form the applicator pad measuring 2.75″×3.5″. The pad was then sealed within a barrier overwrap package, which was prepared for use by removing a portion of the overwrap to expose the active surface. The portion of the overwrap, which was not removed, forms a band for the user's hand to hold the pad, before and during use, without having to touch the active surface.

In field tests, a single antiperspirant pad of this type, proved to be a more convenient and efficient means for evenly applying an effective amount of antiperspirant cream to the underarms.

I claim:

1. A disposable substance delivery applicator comprising:
(a) an impervious backing sheet,
(b) a three-dimensional core layer, wherein said core layer having a first surface side and a second surface side, said first surface side having openings leading to internal cavities in the core layer, said second surface being sealed together with said impervious backing sheet during manufacture, said internal cavities capable of containing a substance for delivery and said substance, when present, having a level internal to said cavities such that said substance is protected from inadvertent contact with external surfaces prior to activation,
(c) a microporous cover sheet sealed to the said first surface of the three dimensional core layer, said microporous cover sheet being capable of directing flow outwardly from the said cavities in the said core layer to the target surface, and
(d) optionally an impervious overwrap placed over the said flow directing cover sheet.

2. A disposable substance delivery applicator comprising:
(a) an impervious backing sheet,
(b) a three-dimensional core layer, wherein said core layer having a first surface side and a second surface side, said first surface side having openings leading to internal cavities in the core layer, said second surface being sealed together with said impervious backing sheet during manufacture, said internal cavities containing a substance for delivery and said substance having a level internal to said cavities such that said substance is protected from inadvertent contact with external surfaces prior to activation,
(c) a substance having substantial resistance to flow prior to delivery to a target surface, occupying the space in said internal cavities in said three dimensional core layer, said substance remaining protected until said three-dimensional core layer is sufficiently deformed into a substantially two-dimensional structure and said substance is thereby exposed to contact a surface of an object without compliance of said surface and contact of the substance with the user's fingers being necessary, said substance having an effective flowability upon activation which permits said substance to be liberated from said location and dispensed onto said surface of said object,
(d) a microporous cover sheet sealed to the said first surface of the three dimensional core layer, said microporous cover sheet being capable of directing flow outwardly from the said cavities in the said core layer to the target surface, and
(e) optionally an impervious overwrap placed over the said flow directing cover sheet.

3. The disposable substance delivery applicator according to claim 2 wherein said internal cavities are in the form of cone-shaped reservoirs.

4. The disposable substance delivery applicator according to claim 2 wherein said internal cavities are in the form of honeycomb-shaped reservoirs.

5. The disposable substance delivery applicator according to claim 2 wherein said activation is through the application of pressure on the said applicator during use.

6. The disposable substance delivery applicator according to claim 2 wherein said substance is in the form of a viscous fluid.

7. The disposable substance delivery applicator according to claim 2 wherein said substance is in the form of a powder.

8. The disposable substance delivery applicator according to claim 7 wherein said activation is through first wetting and then application of pressure on the said applicator during use.

9. The disposable substance delivery applicator according to claim 2 wherein said substance is selected from the group consisting of skin care compositions, moisturizing lotions, tanning lotions, sunscreens, antiperspirants, topical medicaments, cosmetics, lubricants, skin cleansers, hard surface cleaner, polishers, protectants, deodorants, colorants, condiments, adhesives, and combinations thereof.

10. The disposable substance delivery applicator according to claim 1 wherein said applicator is in one of the following forms, hand mitts, finger mitts, gloves, flat pads, tubular pads, wraps, facemasks, and booties.

11. The disposable substance delivery applicator according to claim 2 wherein said applicator is in one of the following forms, hand mitts, finger mitts, gloves, flat pads, tubular pads, wraps, facemasks, and booties.

12. A method of applying an active composition to a targeted skin using the disposable substance delivery applicator according to claim 2 wherein said substance is said active composition and wherein said substance is in the form of a viscous liquid, comprising the steps of
a) removing said overwrap if present,
b) bringing the microporous cover sheet into contact with said skin, and
c) applying pressure to the core layer through the impervious backing sheet by the user's fingers, thereby forcing said active composition onto said skin.

13. A method of applying an active composition to a targeted skin using the disposable substance delivery applicator according to claim 2 wherein said substance is said active composition and wherein said substance is in the form of a powder, comprising the steps of
a) removing said overwrap if present,
b) bringing the microporous cover sheet into contact with said skin, and
c) applying pressure to the core layer through the impervious backing sheet by the user's fingers, thereby forcing said active composition onto said skin.

14. A method of applying an active composition to a targeted skin using the disposable substance delivery applicator according to claim 2 wherein said substance is in the form of a powder and when reconstituted with water, forms said active composition, comprising the steps of
a) removing said overwrap if present,
b) wetting said applicator with water,
c) bringing the microporous cover sheet into contact with said skin, and
d) applying pressure to the core layer through the impervious backing sheet by the user's fingers, thereby forcing said active composition onto said skin.

15. A method of applying an active composition to a targeted hard surface using the disposable substance delivery applicator according to claim 2 wherein said substance is said active composition and wherein said substance is in the form of a viscous liquid, comprising the steps of
a) removing said overwrap if present,
b) bringing the microporous cover sheet into contact with said hard surface, and
c) applying pressure to the core layer through the impervious backing sheet by the user's fingers, thereby forcing said active composition onto said hard surface.

16. A method of applying an active composition to a targeted hard surface using the disposable substance delivery applicator according to claim 2 wherein said substance is said active composition and wherein said substance is in the form of a powder, comprising the steps of
a) removing said overwrap if present,
b) bringing the microporous cover sheet into contact with said hard surface, and
c) applying pressure to the core layer through the impervious backing sheet by the user's fingers, thereby forcing said active composition onto said hard surface.

17. A method of applying an active composition to a targeted hard surface using the disposable substance delivery applicator according to claim 2 wherein said substance is in the form of a powder and when reconstituted with water, is said active composition, comprising the steps of
a) removing said overwrap if present,
b) wetting said applicator with water,
c) bringing the microporous cover sheet into contact with said hard surface, and
d) applying pressure to the core layer through the impervious backing sheet by the user's fingers, thereby forcing said active composition onto said hard surface.

18. A method of manufacturing the disposable substance delivery applicator according to claim 2, comprising the steps of
a) laminating said backing sheet and said core layer together into a roll with said reservoirs facing away from said backing sheet,
b) unwinding said laminated roll with the tops of said reservoirs facing upward,
c) filling the reservoirs with said substance,
d) doctoring off any excess of said substance,
e) overlaying said microporous cover sheet on top of said core layer on the side where the openings of said reservoirs are located,
f) sealing peripherally said microporous cover sheet with said laminate roll, generating a composite structure,
g) die-cutting said composite structure into applicators with desired size and shape, and
h) optionally sealing and packing applicator using said overwrap.
